(19) United States
(12) Patent Application Publication
(10) Pub. No.: US 2025/0268806 A1
(43) Pub. Date: Aug. 28, 2025

(54) PERSONAL CARE COMPOSITION WITH A MULTIFUNCTIONAL INGREDIENT

(71) Applicant: The Procter & Gamble Company, Cincinnati, OH (US)

(72) Inventors: John August WOS, Mason, OH (US); Hilda Andamiche NAMANJA-MAGLIANO, Loveland, OH (US); Jose Carlos GARCIA-GARCIA, Cincinnati, OH (US); Howard David HUTTON, III, Oregonia, OH (US); Isoken Omosefe IGWEKALA-NWEKE, Springdale, OH (US); Amanda Brooke BRODBECK, Cincinnati, OH (US); Ryan Michael WEST, West Chester, OH (US); Dakota James BROCK, West Chester, OH (US); Victor Manuel ARREDONDO, Maderia, OH (US); Sohini Sanjay BHATIA, Madeira, OH (US)

(21) Appl. No.: 19/062,623

(22) Filed: Feb. 25, 2025

Related U.S. Application Data
(60) Provisional application No. 63/719,861, filed on Nov. 13, 2024.
(60) Provisional application No. 63/712,718, filed on Oct. 28, 2024.
(60) Provisional application No. 63/558,670, filed on Feb. 28, 2024.
(60) Provisional application No. 63/557,812, filed on Feb. 26, 2024.

(51) Int. Cl. A61K 8/60; A61Q 5/02

(52) U.S. Cl. CPC A61K 8/604; A61Q 5/02; A61K 2800/596; A61K 2800/70

(57) ABSTRACT

A personal care composition, comprising a mono-rhamno-mono-lipid, a di-rhamno-mono-lipid and a carrier. The combination of rhamno-mono-lipids in a personal cleansing composition yields unexpectedly good lather performance and anti-microbial properties.

### CROSS REFERENCE TO RELATED APPLICATIONS

This application claims the benefit, under 35 U.S.C. § 119 (e), to U.S. Provisional Application Nos. 63/719,861 filed Nov. 13, 2024, 63/712,718 filed Oct. 28, 2024, 63/558,670 filed Feb. 28, 2024, and 63/557,812, filed Feb. 26, 2024, the entire disclosure of which is fully incorporated by reference herein.

### FIELD

The present disclosure generally relates to a personal care composition containing a multifunctional ingredient that provides good lather performance and exhibits antimicrobial properties. More specifically, the present disclosure relates to a hair care composition comprising a mixture of rhamno-mono-lipids that provide good lather and antimicrobial properties.

### BACKGROUND

Personal cleansing compositions, such as shampoos and body washes, are commonly used to restore cleanliness and enhance the appearance of hair and skin. These compositions are necessary due to the accumulation of dirt, sebum, and other environmental contaminants that can negatively impact the look and feel of hair and skin. In recent years, there has been a growing demand for personal care products that incorporate milder cleansers, environmentally friendly ingredients, and fewer overall ingredients.

One area of concern is the use of non-sulfated surfactants. While these surfactants are preferred by some consumers to avoid the perceived harshness of sulfated surfactants, they often exhibit drawbacks in terms of their ability to form coacervates with conditioning polymers, resulting in suboptimal lathering, cleansing, and conditioning performance. Additionally, certain cationic conditioning polymers can introduce instability into sulfate-free surfactant systems, leading to the formation of undesired in situ coacervates that negatively impact product appearance and performance.

Antimicrobial agents and compositions are another area of focus for eliminating or replacing ingredients that may be perceived as environmentally or user unfriendly. Antimicrobial agents are typically added to personal care compositions to prevent or inhibit microbial growth, kill microbes and extend product shelf life. However, environmentally friendly antimicrobial agents can pose challenges when they are not compatible with other ingredients in the composition or they do not satisfy the strict microbiological safety requirements for personal care compositions.

Glycolipid surfactants, such as rhamnolipids and sophorolipids, have recently gained attention for their perceived environmental friendliness. However, they have seen limited use in personal care compositions due, at least in part, to concerns about inferior foaming and cleaning performance. For example, U.S. Pat. No. 10,292,924 discloses a need for a cleansing composition containing a rhamnolipid that has good foam properties. The cleansing composition of U.S. Pat. No. 10,292,924 purportedly addresses this problem by providing a high concentration of di-rhamnolipids relative to mono-rhamnolipids. In contrast, European Patent No. 2,410,039 discloses a cleaning composition characterized by a ratio of mono-rhamnolipid to di-rhamnolipid of 95:5 to 45:55. EP 2410039 indicates that rhamnolipids with two shorter fatty acids are more active in reducing surface tension and as an emulsifier than rhamnolipids with a single fatty acid chain.

Thus, there is a need for improved personal cleansing compositions that strike a balance between mildness, environmental friendliness, effective cleaning and foaming properties, and antimicrobial efficacy. Accordingly, it would be desirable to provide a personal cleansing composition that includes a rhamnolipid surfactant that is perceived by consumers as environmentally friendly and exhibits good foaming and cleaning properties. It would also be desirable to reduce the number of ingredients, especially those perceived as environmentally or user unfriendly, in a personal composition without sacrificing performance, stability or antimicrobial safety.

### SUMMARY

Disclosed herein is a personal care composition, comprising a preservative system comprising a mono-rhamno, mono-lipid of structure I:

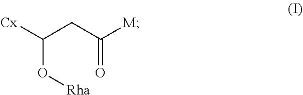

and
a di-rhamno, mono-lipid of structure II:

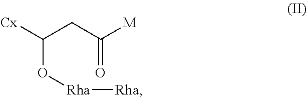

wherein:
Rha is rhamnose,
Cx is a C4-C22 alkyl, aryl, heteroalkyl, heteroaryl, unsaturated alkenyl, or unsaturated heteroalkenyl, and
M is a OH, alkyl, heteroalkyl, aryl, heteroaryl, hetero arylalkyl, arylalkyl, tauryl, O—X+, wherein X+ is a cation, or O—R1, wherein R1 is selected from an alkyl, branched alkyl, and cyclic alkyl, and stereoisomers thereof.
   The composition also includes a carrier, which may be aqueous or anhydrous.

### BRIEF DESCRIPTION OF THE DRAWINGS

FIG. 1 shows exemplary rhamnolipid structures.

FIG. 2 shows an exemplary route for synthesizing rhamno-mono-lipids.

FIG. 3. is a chart illustrating the lather benefit of a rhamno-mono-lipid mixture.

FIG. 4 is a chart illustrating the synergistic foam benefit provided by a mixture of rhamno-mono-lipids and rhamno-di-lipids.

### DETAILED DESCRIPTION OF THE INVENTION

Certain rhamnolipids are known to have antimicrobial activity and generate good lather during use. However, commercially available rhamnolipids for use in mass produced personal care compositions such as shampoos, conditioners and body washes are primarily rhamno-di-lipids, which is to say they have two lipid tails because it generally believed that rhamno-di-lipids are better surfactants than rhamno-mono-lipids. It has now been surprisingly discovered that certain rhamno-mono-lipids provide better anti-microbial efficacy than rhamno-di-lipids without undesirably impacting the lather properties of the composition.

Reference within the specification to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

All ingredient percentages described herein are by weight of the cosmetic composition, unless specifically stated otherwise, and may be designated as “wt %.” All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 25° C. and at ambient conditions, where “ambient conditions” means conditions under about 1 atmosphere of pressure and at about 50% relative humidity. All numeric ranges are inclusive of narrower ranges, and delineated upper and lower range limits are interchangeable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods. As used in the description and the appended claims, the singular forms “a,” “an,” and “the” are intended to include the plural forms as well, unless the context clearly indicates otherwise.

### Definitions

“About” modifies a particular value by referring to a range of plus or minus 20% or less of the stated value (e.g., plus or minus 15% or less, 10% or less, or even 5% or less). “Antimicrobial” means a material that prevents or inhibits the growth of and/or kills microorganisms (e.g., bacteria or fungi).

“Apply” or “application,” as used in reference to a composition, means to apply or spread the composition onto a human keratinous surface such as the skin or hair.

“Cleansing composition” refers to a personal care composition intended for use in cleaning a bodily surface. Some non-limiting examples of cleansing compositions are shampoos, conditioners, conditioning shampoos, shower gels, liquid hand cleansers, facial cleansers, and the like.

“Personal care composition” is meant a product, which in the ordinary course of usage is applied to or contacted with a body surface to provide a beneficial effect such as, for example, improving appearance, cleansing, and odor control. Body surface includes skin, hair, teeth, or nails. Some non-limiting examples of personal care compositions include oral care compositions, (e.g., dentifrice, mouth rinse, mouth spray, lozenge, chewable tablet, chewing gum, teeth whitening strips, floss and floss coatings, breath freshening dissolvable strips, denture care products, and denture adhesive products), shave care compositions (e.g., after shave gels and creams, pre-shave preparations, shaving gels, creams), cough and cold compositions, leave-on skin lotions and creams, shampoos, body washes, hair conditioners, hair dyeing and bleaching compositions, styling mousses, shower gels, bar soaps, hand soaps, antiperspirants, deodorants, depilatories, lipsticks, foundations, mascara, sunless tanners and sunscreen lotions, feminine care compositions and absorbent articles, baby care compositions and absorbent articles.

“Substantially free of” means a composition or ingredient comprises less than 3% of a subject material, by weight of the composition or ingredient (e.g., less than 2%, less than 1% or even less than 0.5%). “Free of” means a composition or ingredient contains 0% of a subject material.

“Synergy” and variations thereof mean that the effect provided by a combination of two or more materials (e.g., a combination of rhamno-di-lipid and rhamno-mono-lipid and/or a combination of rhamno-mono-lipids with different carbon chain lengths) is more than the additive effect expected for these materials.

### Personal Care Composition

The personal care compositions herein include a multifunctional rhamnolipid that provides lather/cleansing, conditioning and antimicrobial properties. The rhamnolipid may be present in the composition at 0.5% to 30% (e.g., 1% to 25%, 2% to 20%, 3% to 15%, or 5% to 10%), based on the weight of the personal care composition. The personal care composition may, optionally, include an additional surfactant (e.g., anionic, non-ionic and/or amphoteric surfactant) and/or other ingredients commonly found in compositions of the type described. In some aspects, the personal care composition may contain less than 10% of sulfated surfactants such as sodium lauryl sulfate and sodium laureth sulfate (e.g., less than 8%, 7%, 5%, 3%, 2%, 1% or even 0%).

The personal care compositions herein may be provided in various product forms such as solutions, suspensions, shampoos, conditioners, lotions, creams, gels, toners, sticks, sprays, aerosols, ointments, cleansing liquid washes, solid bars, pastes, foams, mousses, shaving creams, wipes, strips, patches, hydrogels, film-forming products, facial and skin masks (with and without insoluble sheet), and the like. The composition form may follow from the particular dermatologically acceptable carrier chosen. In some aspects, the personal care compositions described herein may include a dispersed gel network phase that provides a milder, but effective, cleansing benefit to soiled hair in combination with a detersive glycolipid surfactant.

In some aspects, the compositions herein are free of or substantially free of thickeners. For example, the composition may contain less than 1% (e.g., 0% to 0.8%, 0.05% to 0.5%, or even 0.1% to 0.3%) of an inorganic salt thickener such as sodium chloride, potassium chloride, sodium sulfate, ammonium chloride, sodium bromide, combinations of these and the like. In sulfate-free cleansing compositions, inorganic salt can introduce instability to the composition by aiding in the formation of an undesirable coacervate between anionic surfactants and cationic polymers prior to the intended use of the composition. Inorganic salt and other thickeners can undesirably impact the rheological and performance properties of the present composition as well as the consumer-perceived quality of the product.

Other examples of thickeners that may be excluded or substantially excluded from the present compositions include homopolymers based on acrylic acid, methacrylic acid or other related derivatives (e.g., polyacrylate, polymethacrylate, polyethylacrylate, and polyacrylamide), acrylic copolymers or methacrylate copolymers (e.g., acrylates/C10-C30 alkyl acrylate crosspolymer), crosslinked acrylic polymers (e.g., carbomer), hydrophobically modified cellulose derivatives; hydrophobically modified, alkali swellable emulsions (e.g., hydrophobically modified polyacrylates, polyacrylic acids, polyacrylamides and polyethers) cellulose and its derivatives (e.g., microcrystalline cellulose, carboxymethylcelluloses, methylcellulose, ethylcellulose), guar and its derivatives (e.g., hydroxypropyl guar, and hydroxypropyl guar hydroxypropyl trimonium chloride), polyethylene oxide, polypropylene oxide, polyvinylpyrrolidone, polyvinyalcohol and its derivatives, polyethyleneimine and its derivatives, silicas (e.g., fumed silica, precipitated silica, and silicone-surface treated silica), water swellable natural polymers (e.g., xanthan gum, guar gum, arabia gum, carob gum and locust bean gum), sorbitol, karaggenan, pectin, agar, starch (from rice, corn, potato, wheat, etc) and starch derivatives (e.g., carboxymethyl starch, methylhydroxypropyl starch). Still further examples of thickeners, rheology modifiers and suspending agents that may be excluded from the present composition are disclosed in U.S. Pat. No. 10,258,555.

It is to be appreciated that embodiments in which the present compositions include materials commonly used as rheology modifiers (thickeners, etc.) are contemplated herein. In such embodiments the material may be included to provide a function or benefit other than thickening or may be specifically selected because it does not undesirably interact with other ingredients in the composition (form coacervate prior to use, etc.).

### RHAMNOLIPID

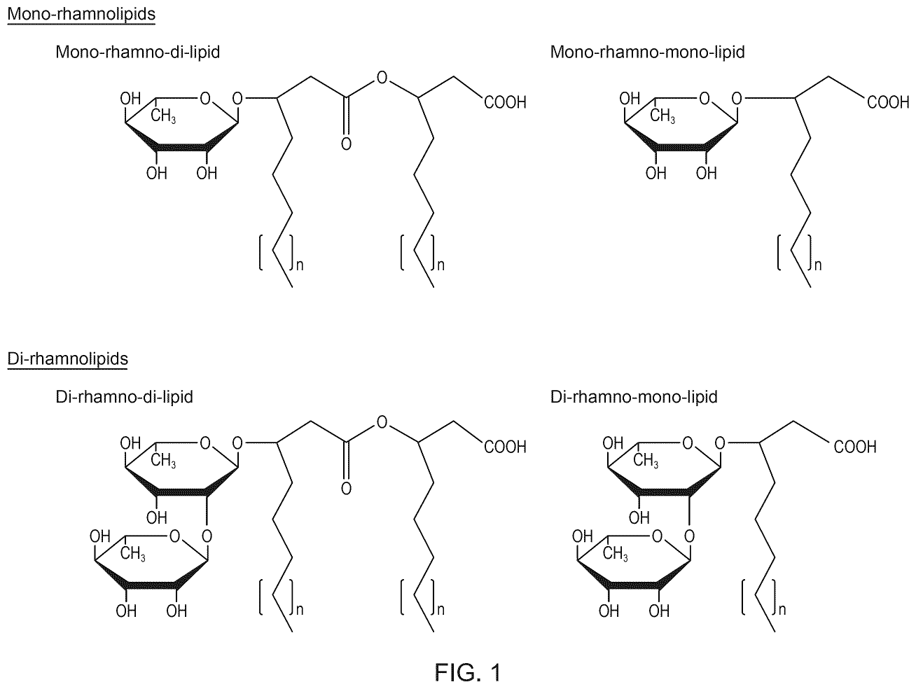

Rhamnolipids include a glycosyl head group (rhamnose) and a fatty acid tail (lipid). The resulting molecular structure imparts amphiphilic properties essential for their effectiveness as surfactants. The two main classes of rhamnolipids are mono-rhamnolipids and di-rhamnolipids, which consist of one or two rhamnose head groups, respectively. Each class of rhamnolipid can have either one or two lipid tails (mono-lipids or di-lipids, respectively), as exemplified in FIG. 1. A more general description of glycolipid surfactants can be found in U.S. Publication No. 2023/0320961.

The personal care compositions herein include a mixture of rhamno-mono-lipids for delivering a lathering/cleansing benefit and an antimicrobial benefit. In some instances, the rhamnolipid surfactant system may also deliver a conditioning benefit. Multifunctional ingredients like the rhamnolipids herein can be used to formulate personal care compositions that have fewer ingredients, which is desired by some consumers.

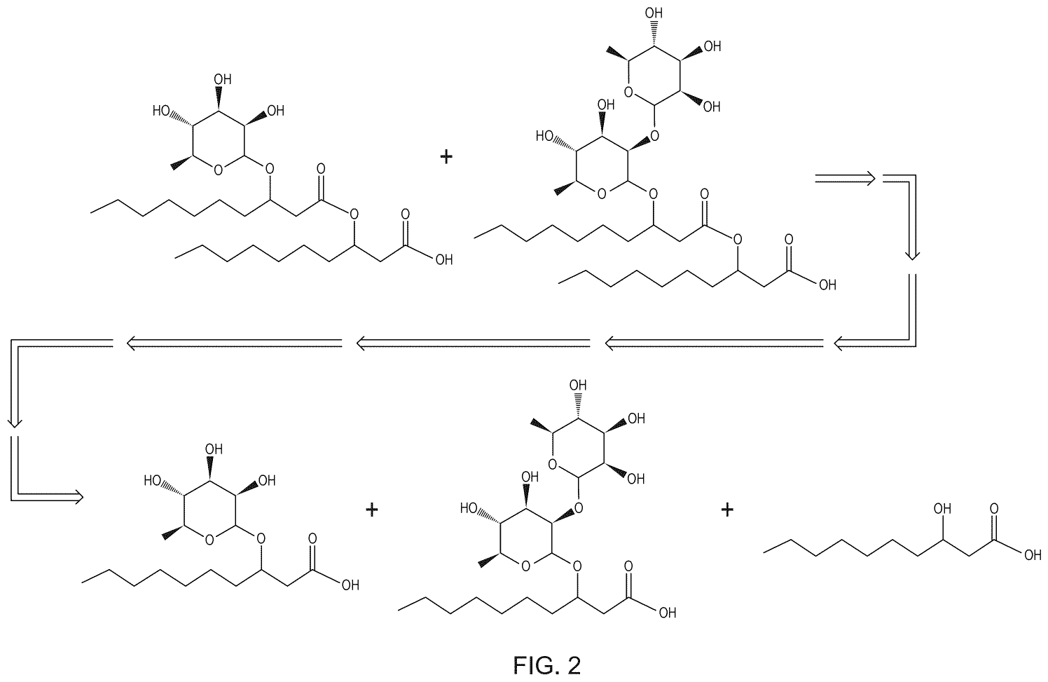

In some instances, the rhamno-mono-lipid mixture can be made using a semi-synthetic method that involves performing a base hydrolysis on a rhamno-di-lipid compound (e.g., Rheance® One brand rhamnolipids from Evonik Industries AG, Essen, Germany). Possible bases include sodium hydroxide (NaOH), potassium hydroxide (KOH), calcium hydroxide (Ca(OH)₂), cesium hydroxide (CsOH), magnesium hydroxide (Mg(OH)₂), ammonium hydroxide (NH₄OH), and alkylamine containing bases such as triethylamine (Et₃N). A semi-synthetic approach for hydrolyzing rhamno-di-lipids is exemplified in FIG. 2.

The rhamno-mono-lipids resulting from the hydrolysis of a rhamno-di-lipid species may include 25% or more, based the total weight of the rhamnolipids, of rhamno-mono-lipids (e.g., greater than or equal to 30%, 35%, 40%, 45%, 50%, 55%, 60%, 65%, 70%, 75%, 80%, 85%, 90%, 95%, 96%, 97%, 98%, 99%, or even 100%). These exemplary amounts of rhamno-mono-lipid species may be any combination of di-rhamno-mono-lipid and mono-rhamno-mono-lipid species, including only a single species of rhamno-mono-lipid. In some instances, the weight ratio of mono-rhamno-mono-lipid to di-rhamno-mono-lipid in the composition may range from 1:10 to 10:1 (e.g., 1:5 to 5:1, 1:4 to 4:1, 1:3 to 3:1, 1:2 to 2:1 or even about 1:1).

The reaction product of di-rhamno-di-lipid hydrolysis may be free of or substantially free of di-lipid species (i.e., di-rhamno-di-lipid and mono-rhamno-di-lipid species). In other words, the hydrolysis reaction may completely convert the di-lipid species to mono-lipid species. In some instances, the di-lipid species may not be completely hydrolyzed, and it may be desirable to remove the di-lipid species using conventional methods known in the art. In some instances, the preservative system herein may include less than 25% of a di-lipid species, based on the total weight of the rhamnolipids (e.g., less than 20%, 15%, 10%, 5%, 4%, 3%, 2%, 1% or even 0%).

Some non-limiting examples of methods for making the rhamno-mono-lipids herein, including suitable methods for hydrolyzing rhamno-di-lipids, are disclosed in U.S. Provisional Ser. Nos. 63/558,670, 63/557,812 and 63/641,630.

The reaction product resulting from the hydrolysis of the di-rhamno-di-lipid species includes compounds (a), (b) and (c), and all stereo isomers thereof, as shown below:

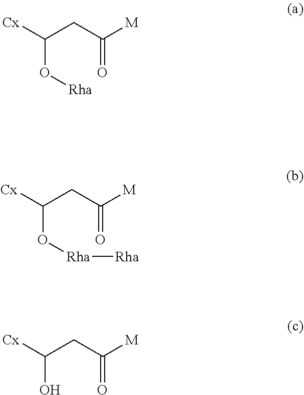

wherein Rha is rhamnose;
wherein each Cx is independently selected from an alkyl, aryl, heteroalkyl, heteroaryl, unsaturated alkenyl, and unsaturated heteroalkenyl;
wherein each Cx independently has a carbon chain length from 4 to 22 (e.g., 5 to 13, 10 or 12);
wherein each M is independently selected from OH; O—X+wherein X+is a cation; OR1; alkyl, heteroalkyl; aryl; heteroaryl; hetero arylalkyl; arylalkyl; and tauryl;
wherein R1 is selected from an alkyl, branched alkyl, and cyclic alkyl.

Some nonlimiting examples of mono-rhamno mono-lipid that may be suitable for use herein include 3-(((2R,3R,4R,5R,6S)-3,4,5-trihydroxy-6-methyltetrahydro-2H-pyran-2-yl)oxy)hexadecanoic acid, 3-(((2R,3R,4R,5R,6S)-3,4,5-trihydroxy-6-methyltetrahydro-2H-pyran-2-yl)oxy)octenoic acid, 3-(((2R,3R,4R,5R,6S)-3,4,5-trihydroxy-6-methyltetrahydro-2H-pyran-2-yl)oxy)decenoic acid, 3-(((2R,3R,4R,5R,6S)-3,4,5-trihydroxy-6-methyltetrahydro-2H-pyran-2-yl)oxy)octanoic acid, 3-(((2R,3R,4R,5R,6S)-3,4,5-trihydroxy-6-methyltetrahydro-2H-pyran-2-yl)oxy)tetradecanoic acid, 3-(((2R,3R,4R,5R,6S)-3,4,5-trihydroxy-6-methyltetrahydro-2H-pyran-2-yl)oxy)decanoic acid, 3-(((2R,3R,4R,5R,6S)-3,4,5-trihydroxy-6-methyltetrahydro-2H-pyran-2-yl)oxy)dodecanoic acid, 3-(((2R,3R,4R,5R,6S)-3,4,5-trihydroxy-6-methyltetrahydro-2H-pyran-2-yl)oxy)dodecen-dienoic acid, and stereoisomers thereof.

Some non-limiting examples of di-rhamno mono-lipid that may be suitable for use herein include 3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)hexadecanoic acid, 3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)tetradecanoic acid, 3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)decenoic acid, 3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy) tetradecenoic acid, 3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)decanoic acid, 3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)octanoic acid, and 3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)dodecanoic acid, and stereoisomers thereof.

The personal care compositions herein may free of or substantially free of rhamno-di-lipids. However, in some instances it may be desirable to include a rhamno-di-lipid (mono-rhamno and/or di-rhamno) because it has been surprisingly discovered that the amount of rhamno-di-lipid and/or ratio of rhamno-mono-lipid to rhamno-di-lipid can be tailored to achieve synergistic lather benefits, as discussed in more detail in the examples below, without sacrificing anti-microbial efficacy. In some instances, the composition may include a ratio of rhamno-di-lipid to a total amount of rhamn-mono-lipid of less than 3:1 (e.g., 4:1, 5:1, 6:1, 7:1, 8:1, 9:1 or even 10:1 or less).

### Aqueous Carrier

The personal care compositions herein can include an aqueous carrier. The level and species of the carrier can be selected according to the compatibility with other components and other desired characteristic of the product. The carrier may include water include and/or water miscible liquids such as lower alkyl alcohols. The lower alkyl alcohols can be monohydric alcohols having 1 to 6 carbons. The aqueous carrier may be present at 10% to 95% based on the weight of the preservative system. Of course, it is to be appreciated that the preservative system may consist of or consist essentially of components (a), (b) and, optionally, (c) described above.

### Additional Components

The preservative systems herein may optionally include additional components suitable for use in personal care compositions. Some non-limiting examples of additional ingredients include, for example, other antimicrobial agents, active agents for providing a hair or scalp benefit, additional surfactants, humectants, emollients, thickeners, fragrances, stabilizers, colorants, and antioxidants. Such agents and the amounts in which they may be incorporated would be known to those of ordinary skill in the art. For example, an optional additional ingredient may be present at 0.1% to 30% (e.g., 0.5% to 25%, 1% to 20%, 3% to 15%, or 5% to 10%).

In some aspects, the personal care composition may be free of or substantially free of antimicrobial agents or other ingredients that do not meet a particular sustainability standard or naturally derived ingredient standard such as, for example, EWG VERIFIED™, Whole Foods® unacceptable ingredients list, and “risk-free” (green dot) by the Yuka® Application. Some non-limiting examples of antimicrobial agents that may not be suitable for use herein include isothiazolinones (e.g., 5-chloro-2-methyl-4-isothiazolin-3-one and 2-methyl-4-isothiazolin-3-one, commercially available as Kathon™CG from Dow®), benzyl alcohol, phenoxyethanol, cyclohexylglycerin, parabens, and ethylenediaminetetraacetic acid (EDTA) and salts thereof.

The personal care composition herein may optionally include a co-surfactant selected from anionic surfactants, amphoteric surfactants, zwitterionic surfactants, non-ionic surfactants and combinations of these. Some non-limiting examples of anionic surfactants include non-sulfated anionic surfactants such as isethionates, carboxylates, sulfonates (e.g., alpha olefin sulfonates, linear alkylbenzene sulfonates, alkyl glyceryl sulfonates, sodium laurylglucosides hydroxypropylsulfonate), sulfosuccinates, sulfoacetates, sulfolaurates, amino acid-based surfactants (e.g., glycinates, taurates, alaninates, glutamates), lactate-and lactylate-based surfactants (e.g., sodium lauroyl lactate and sodium lauroyl lactalyte), phosphate ester surfactants and combinations thereof.

Some non-limiting examples of amphoteric and/or zwitterionic surfactants include derivatives of aliphatic secondary and tertiary amines in which one of the aliphatic substituents contains from 8 to 18 carbon atoms and one aliphatic substituent contains an anionic group such as a carboxy, sulfonate, phosphate, or phosphonate group. For example, amphoacetates, amphodiacetates, betaines, amidobetaines (e.g., cocamidopropyl betaine and lauramidopropyl betaine), amidosulfobetaines, propionates, sultaines, hydroxysultaines, and combinations thereof.

Some non-limiting examples of non-ionic surfactants include glyceryl esters of alkanoic acids, polyglyceryl esters of alkanoic acids, propylene glycol esters of alkanoic acids, sorbitol esters of alkanoic acids, alkanolamides, alkoxylated amides, alkyl glycosides, alkyl polyglucosides acyl glucamides, amine oxides and combinations thereolf. Some particularly suitable examples of non-ionic surfactants include cocamide, cocamide MEA, PPG-2 cocamide, PPG-2 hydroxyethyl cocamide, PPG-2 hydroxyethyl isostearamide, lauroyl/myristoyl methyl glucamide, capryloyl/caproyl methyl glucamide, cocoyl methyl glucamide, decyl glucoside, coco-glucoside, lauryl glucoside, lauramine oxide, cocamine oxide and combinations thereof.

More specific examples of the optional co-surfactants described above are disclosed in U.S. Publication No. 2019/0105246, U.S. Publication No 2018/0098923, U.S. Pat. No. 9,271,908, PCT Publication No. WO 2020/016097, and Mccutcheon's Emulsifiers and Detergents, 2019, MC Publishing Co.

The optional additional surfactants, when present, may be included in the personal care compositions to provide the desired cleaning and lather performance. Any additional surfactants should be physically and chemically compatible with the other components of the personal care compositions described herein and should not otherwise unduly impair product stability, aesthetics, or performance. In some aspects, additional surfactants may be present in the personal care compositions at 5% to 50% (e.g., 8% to 30%, 9% to 25%, or even 10% to 17%).

### Dispersed Gel Network

The personal care compositions described herein may include a dispersed gel network phase to provide a cleaning and/or conditioning benefit to the composition in combination with the detersive surfactant. A gel network phase can confer a cleaning benefit to the personal care composition through its hydrophobic nature. Specifically, it is believed, without being limited by theory, that the hydrophobic nature of the dispersed gel network allows the gel network to dissolve hydrophobic soils such as oil into the gel network. Once the soils are dissolved into the gel network, the gel network can be rinsed out of the hair or skin.

Suitable dispersed gel networks can be formed by combining a fatty alcohol and a gel network surfactant in a suitable ratio and heating the dispersion to a temperature above the melting point of the fatty alcohol. During the mixing process, the fatty alcohol melts allowing the gel network surfactant to partition and bring water into the fatty alcohol. Mixing of the gel network surfactant and fatty alcohols also changes the isotropic fatty alcohol drops into liquid crystalline phase drops. When the mixture is subsequently cooled below the melt transition temperature of the fatty alcohols, the liquid crystal phase is converted into a solid crystalline gel network. Additional details of suitable gel networks are described in G. M. Eccleston, “Functions of Mixed Emulsifiers and Emulsifying Waxes in Dermatological Lotions and Creams”, Colloids and Surfaces A: Physiochem. and Eng. Aspects 123-124 (1997) 169-182; and by G. M Eccleston, “The Microstructure of Semisolid Creams”, Pharmacy International, Vol. 7, 63-70 (1986), each of which is incorporated by reference herein.

The presence of a gel network in the pre-mix and in a personal care composition can be confirmed by means known to one of skill in the art. For example, X-ray analysis, optical microscopy, electron microscopy, and differential scanning calorimetry can be used to identify a gel network. A suitable x-ray analysis method is described in U.S. Publication No. 2006/0024256.

In some aspects, the scale size of the dispersed gel network in a personal care composition can range from about 10 nm to about 500 nm (e.g., 0.5 μm to 10 μm or 10 μm to about 150 μm).

### Gel Network Fatty Alcohol

The dispersed gel network may include a fatty alcohol (e.g., C10-C40 fatty alcohols) at 0.05% or more by weight of the composition (e.g., 0.05% to about 25%, 0.5% to 20%, or 1% to 8%). The fatty alcohol may be straight or branched chain and can be saturated or unsaturated. As can be appreciated, suitable fatty alcohols can be of natural, vegetable, or synthetic origin. In some aspects, it may be desirable to mix several fatty alcohols to provide a dispersed gel network phase with a melt transition temperature of about 38° C. or greater such as, for example, a mixture of cetyl alcohol and stearyl alcohol at a ratio of between 20:80 and 80:20. Some non-limiting examples of fatty alcohols that may be suitable for use herein include cetyl alcohol, stearyl alcohol, arachidyl alcohol, behenyl alcohol, C₂1 fatty alcohol (1-heneicosanol), C₂₃ fatty alcohol (1-tricosanol), C₂₄ fatty alcohol (lignoceryl alcohol, 1-tetracosanol), C₂₆ fatty alcohol (1-hexacosanol), C₂₈ fatty alcohol (1-octacosanol), C₃₀ fatty alcohol (1-triacontanol), C₂₀₋₄₀ alcohols (e.g., Performacol® 350 and 425 Alcohols, available from New Phase Technologies), C₃₀₋₅₀ alcohols (e.g., Performacol® 550 Alcohol), C₄₀₋₆₀ alcohols (e.g., Performacol® 700 Alcohol), and mixtures thereof.

### Gel Network Surfactant

The gel network phase may include a gel network surfactant at 0.01% to 15% by weight of the composition (e.g., 0.1% to about 10%, 0.2% to about 5%). The gel network surfactant is combined with the fatty alcohol and liquid carrier to form a gel network pre-mix, which can then be added to the other ingredients of the personal care composition.

In some aspects, the total weight of the gel network surfactant and the fatty alcohols is 0.5% to about 15% by weight of the personal care composition (e.g., 1% to 10%). In some aspects, the gel network surfactant may be included in the gel network at a desired weight ratio with respect to the fatty alcohols. For example, the ratio of the fatty alcohols to the gel network surfactant may be 1:5 to 100:1 (e.g., 1:1 to 40:1, 2:1 to 20:1, or even 3:1 to 10:1).

The gel network surfactant can be any suitable anionic, zwitterionic, amphoteric, cationic, and nonionic surfactants that is substantially free of sulfates. The detersive surfactant and the gel network surfactant can be the same or different. In some aspects, the gel network surfactant has a hydrophobic tail group with a chain length of 10 to 40 carbon atoms. The hydrophobic tail group may be alkyl, alkenyl (containing up to 3 double bonds), alkyl aromatic, or branched alkyl.

Mixtures of more than one gel network surfactant can also be used. Some non-limiting examples of gel network surfactants are disclosed in U.S. Publication No. 2006/0024256.

### Liquid Carrier for the Gel Network

In some aspects, the dispersed gel network phase may include a suitable liquid carrier at 0.05% to 95% by weight of the personal care composition. The liquid carrier can be water or another suitable solvent. The carrier and the gel network surfactant may be selected to work together to swell the fatty alcohol, which leads to the formation and stability of the gel network phase. A suitable solvent is any that can be used in the place of or in combination with water in the formation of the gel network phase. In some aspects, the liquid carrier can be substantially free of solvents other than water. In some aspects, the liquid carrier for the dispersed gel network phase can be included at a weight ratio of about 1:1 with the fatty alcohol of the dispersed gel network phase.

### Cationic Polymer

The personal care compositions herein may include 0.05%-3% of a cationic polymer (e.g., 0.1-2%, or even 0.2-0.8%) to provide a conditioning benefit (e.g., improved appearance, feel or deposition benefits) to hair or skin. The cationic polymer can have a weight average molecular weight of 100 kDa to 5 MDa (e.g., 500 kDa to 4 MDa, 1 MDa to 3 MDa, or even 1.2 MDa to 2 MDa) and a charge density of 0.4 meq/g to 12 meq/g (e.g., 0.4 to about 2, from about 0.7 to about 2, and from about 0.6 to about 1.6. The charge densities can be measured at the pH of intended use of the personal care composition, which can be pH 3 to pH 9 (e.g., pH 4-8 or pH 4.5-6.5). The cationic polymer should be selected to form a coacervate with the anionic surfactant, and optional co-surfactant, during the intended use of the personal care composition.

The cationic polymers may include cationic, nitrogen-containing moieties such as quaternary ammonium or cationic protonated amino moieties. The cationic protonated amines can be primary, secondary, or tertiary amines, depending on the particular species and the selected pH of the composition. Anionic counterions can be used in association with the cationic polymers, as long as the polymers remain soluble. Examples of suitable counterions include halide counterions (e.g., chloride, fluoride, bromide, iodide). Some non-limiting examples of cationic polymers that may be suitable for use herein are described in U.S. Publication No. 2023/0320961.

### Carrier

The composition may optionally include 20-95% of an aqueous carrier such as water and/or a water miscible solvent. The type and amount of aqueous carrier should be selected to provide the composition with the desired rheological properties. The liquid carrier can be water with, e.g., less than 5%, 3%, 1%, 0.5% or even 0% miscible organic solvent. Some nonlimiting examples of organic solvents include lower alkyl alcohols (e.g., ethanol and isopropanol) and polyhydric alcohols (e.g., propylene glycol, hexylene glycol, glycerin, and propane diol).

### Methods

### Lather Height and Creaminess

The foaming potential of shampoos does not directly influence the physical behavior of hair fibers. However, shampoo foam can influence a user's perception of hair characteristics. In some instances, it may be helpful to distinguish lather from foam for shampoo evaluation. In particular, foam can generically refer to a mass of gas bubbles in a liquid film matrix, whereas lather more specifically refers to a type of foam formed during shampooing and other processes, wherein the foam consists of small bubbles that are densely packed, thus resisting flow.

This method provides a way to simulate the lather produced by surfactants when used on hair under typical shampooing conditions and quantify certain lather properties. Oil (e.g., sebum) and dirt are the two most common contaminates found on hair that can undesirably affect the lather properties of a shampoo. Thus, this method can be used to evaluate the effect of oil or dirt on lather properties.

### Lather Height (Oil) and Creaminess

100 ml of water (at 100° F.) is placed in a suitable blender (e.g., KitchenAid KSB560CU1 brand food mixer or equivalent), followed by 2 mL of the test composition and 1 mL of extra virgin olive oil. Blend the mixure on “stir” for 30 seconds and record the height of the lather.

To assess creaminess, the lather is poured into a suitable bowl and is visually inspected. Based on the visual inspection, lather creaminess is rated from 0 to 5, where 0 is not at all creamy (bad) and 5 is the extremely creamy (good).

### Lather Height (Dirt)

300 mL of water (at 100° F.) is placed in a suitable blender (e.g., KitchenAid KSB560CU1 brand food mixer or equivalent), followed by 3 mL of the test composition and 2 grams of potting soil (e.g., MIRACLE GRO brand potting soil or equivalent). Blend the mixture on “stir” for 15 seconds and record the height of the lather.

### Wet Hair In-Lab Screening (ILS)

This method can be used to determine the cleaning and/or conditioning properties of the personal care compositions herein. In this method, a 20 g hair switch of Caucasian Low Lift Hair Tresses (International Hair Importers and Products, Inc.; Glendale, NY) is wetted with water, treated with a personal care composition and subject to testing in an In-Lab Screening (ILS) sink. The sink has a salon spray head/hose that is held in place but can be directed to run water over a hair tress that hangs from a rod placed over the sink. The tress can be moved in and out of the water as necessary. The water is maintained at a temperature of 38° C. and a flow rate of 5.7 liters per minute. The testing is as follows:
Calibrate ILS sink to 38° C.
Hang the hair tress switch on rod in sink.
Wet hair thoroughly for 30 seconds. Squeegee the hair tress switch once using your index and middle finger (“scissor fingers) from top to bottom to remove excess water. (“Squeegee” means to clamp the tress at the top in between your index and middle finger and stroke down once to remove water.)
Apply 0.1 g/g (product/hair) of the test composition to the front of the switch, from top to bottom.
Milk the hair tress switch for 15 seconds, then flip the bottom of the switch to the top and milk for another 15 seconds. (“Milk” means to grab the top of the tress and stroke it downward while alternating hands to create lather.)
Evaluate Lather Creaminess (look and feel, after 30 seconds of lathering). Scale: 0=No Creaminess-10=Extremely Creamy
Evaluate Lather Combing (with lather still in switch). Using the lowest pressure possible, place comb all the way through the hair (starting at the top) and using a minimal amount of force (comb from top to bottom) comb through hair switch. Scale: 0=hard to comb-10=easy to comb.
· Rinse for 30 seconds (while lightly milking the switch).
· Squeegee the hair switch tress once with scissor fingers.
· Evaluate Slippery Feel. Squeegee once and assess feel. Scale: 0=No Slip-10=Extremely Slippery.
Evaluate Clean Feel Post Rinse. Stroke the hair from top to bottom between the thumb and two fingers with medium pressure. Gauge how clean or dirty the hair feels. Scale: 0=Low (Dirty)−10=High (Clean).
Evaluate Post Rinse Comb. Using the lowest pressure possible, placing comb (wide tooth side) all the way through the hair (front to back), and using a minimal amount of force (top to bottom) comb through hair switch. Scale: 0=Hard−10=Easy.

### Microbial Susceptibility Test (MST)

Organisms are prepared for Microbial Susceptibility Testing as follows: Bacteria, including Escherichia coli (ATCC 190 8739, American Type Culture Collection, Manassas, Virginia, USA), Staphylococcus aureus (ATCC #6538, American Type Culture Collection, Manassas, Virginia, USA), Pseudomonas aeruginosa (ATCC #9027, American Type Culture Collection, Manassas, Virginia, USA), Burkholderia cepacia (ATCC 190 25416, American Type Culture Collection, Manassas, Virginia, USA), as well as environmental isolates of Klebsiella pneumoniae, Enterobacter gergoviae and Serratia marcescens, are streaked on Tryptic Soy Agar (TSA, Becton Dickinson DIFCO™ Tryptic Soy Agar, Franklin Lakes, NJ, USA) and incubated at 30-35° C. for 18-24 hrs. Candida albicans (ATCC #10231, American Type Culture Collection, Manassas, Virginia, USA) is streaked on Sabouraud Dextrose Agar (SDA, Neogen, Lansing, MI, USA) and incubated at 20-25° C. for 44-52 hrs while Aspergillus brasiliensis (ATCC #16404, American Type Culture Collection, Manassas, Virginia, USA) is streaked on SDA and incubated in a dark 20-25° C. chamber for 6-10 days until dense dark sporulation is observed. Organism suspensions are prepared by transferring confluent growth to saline (0.85% NaCl) or saline with 0.05% Tween 80(polysorbate 80) (A. brasiliensis only) and turbidometrically adjusted to a target concentration of 10⁷-10⁸ CFU/ml.

Bacterial microbial susceptibility is tested as follows: A bacterial pool (mixture in equal volumes) of challenge organisms, is used in this test and prepared such that the final concentration is approximately 6-8 log cfu/ml. This inoculum is added at a ratio of 1% v/w to product and inoculated products are incubated at 20-25° C. for up to 7 days. Organism survival is measured during and at the end of the incubation period by neutralizing an aliquot of inoculated sample in Modified Letheen Broth containing 1.5% polysorbate 80 and 1% Lecithin (MLBTL). Successive dilutions are transferred into petri dishes containing Modified Letheen Agar with 1.5% Tween 80(polysorbate 80), and the agar plates are incubated at least 2 days at 30-35° C. Bacterial colony forming units (cfu) are then enumerated, and a bacterial log reduction from the starting log cfu/g challenge level is reported. Greater log cfu/g reduction values indicate greater anti-bacterial robustness.

Fungal microbial susceptibility is tested as follows: Candida albicans and Aspergillus brasiliensis are mixed in equal volumes such that the concentration of the fungal pool is approximately 6-8 log cfu/ml. This inoculum is added at a ratio of 1% v/w to product and inoculated product is incubated at 20-25° C. for up to 14 days. Organism survival is measured during and at the end of the incubation period by neutralizing an aliquot of inoculated sample in Modified Letheen Broth containing 1.5% polysorbate 80 and 1% Lecithin (MLBTL). Successive dilutions are transferred into petri dishes containing Sabouraud Dextrose Agar, and the agar plates are incubated at least 2 days at 30-35° C. Fungal colony forming units (cfu) are then enumerated, and a fungal log reduction from the starting log cfu/g challenge level is reported. Greater log cfu/g reduction values indicate greater anti-fungal robustness.

Desired levels of microbial susceptibility are provided in Table 1.

TABLE 1
Microbial Susceptibility

| | Day 2 | Day 7 | Day 14 |
|---|---|---|---|
| Bacteria | ≥2 log | ≥3 log | — |
| | reduction | reduction | |
| Mold/yeast | — | — | ≥2 log |
| | | | reduction |

### EXAMPLES

### Example 1: Formulations

Examples of inventive personal care compositions are provided in Tables 2A and 3A. The example compositions are made using conventional methods. For Table 2A, Control Personal Cleansing Composition 1 can be formed by the following process. DI water is added to a mixing vessel and heated to 75° C.±3° C. while agitating. Sodium Cocoyl Isethionate (SCI) is added to the mixing vessel, and the mixing continues until the SCI has fully dissolved (with no visible particles remaining and batch is clear). After the SCI has fully dissolved, the following materials are added to the mixing vessel: Alkyl Amidopropyl Betaine and Sodium Lauroyl Sarcosinate. The vessel contents are mixed for at least 10 minutes. The batch is then cooled to <35° C. A Polyquaternium-10 slurry is made with water, which is immediately added to the mixing vessel and mixed for 10 minutes. Perfume is then added and mixed in the mixture for at least 2 minutes. Citric Acid is used to titrate the mixture until a pH of 5.5 to 6.0 is reached. DI water is added to bring the final volume to 100%. The mixture is mixed for at least 10 minutes until homogeneity is achieved.

Control Personal Cleansing Compositions 2 can be formed by the following process. DI water is added to a mixing vessel while agitating. The following material is then added to the mixing vessel: Alkyl Amidopropyl Betaine. The vessel contents are mixed for at least 10 minutes.

A Polyquaternium-10 slurry is made with water, which is immediately added to the mixing vessel and mixed for 10 minutes. Perfume is then added and mixed in the mixture for at least 2 minutes. Citric Acid is used to titrate the mixture until a pH of 5.5 to 6.0 is reached. DI water is added to bring the final volume to 100%. The mixture is mixed for at least 10 minutes until homogeneity is achieved.

Comparative Personal Cleansing Compositions 1-6 and Inventive Personal Cleansing Compositions 1-6 can be formed by the following process. Deionized water is added to a mixing vessel while agitating. The following materials are then added to the mixing vessel: Alkyl Amidopropyl Betaine and Rhamnolipids. The vessel contents are mixed for at least 10 minutes. A Polyquaternium-10 slurry is made with water, which is immediately added to the mixing vessel and mixed for 10 minutes. Perfume is then added and mixed in the mixture for at least 2 minutes. Citric acid is used to titrate the mixture until a pH of 6.8 to 7.2 is reached. Deionized water is added to bring the final volume to 100%. The mixture is mixed for at least 10 minutes until homogeneity is achieved.

The Inventive Examples, comprising the inventive hydrolyzed rhamnolipids cocktail, have significant lather volume and creaminess improvements over the Comparative Examples that comprise commercial rhamnolipid controls when rhamnolipids are used as the primary anionic surfactant in sulfate-free shampoo formulations.

The rhamn-di-lipids used in the comparative examples (C1, C2 and C3) were sourced from commercial suppliers, and generally had a ratio of 3.6 wt % Rhamno-mono-lipids/94 wt % Rhamno-di-lipids. The rhamno-mono-lipids in the inventive examples (Inv1, Inv2, and Inv3) were obtained by hydrolyzing a rhamno-di-lipid material. After hydrolysis some of the resulting mono-rhamnolipid was subjected to further processing to remove certain undesired components, such as beta hydroxy fatty acids and/or unreacted rhamno-di-lipids. Inventive compositions 1, 2 and 3 were tested using post-hydrolysis purified rhamno-mono-lipid (designated as “A”) and unpurified rhamno-mono-lipid (designated as “B”). Inventive Composition 4 contained the unpurified rhamnolipid. Control composition 1 (Control 1) is an in-market, sulfate-free shampoo composition. Control composition 2 (Control 2) is an in-market shampoo composition that is free of anionic surfactant. Control 1 and 2 are used to provide a performance benchmark. Example Compositions 5 and 6 (Ex 5 and 6) are provided to show that not all composition containing rhamno-mono-lipids will necessarily provide the desired antimicrobial and lather benefit.

TABLE 2A
Sulfate-free Shampoo Formulations

| | Control | Control | | Inv 1 | | | Inv 2 | | | Inv 3 | |
|---|---|---|---|---|---|---|---|---|---|---|---|
| Component | 1 | 2 | C 1 | A | B | C 2 | A | B | C 3 | A | B |
| Alkyl | 9.8 | 9.8 | 9.8 | | | | | | | | |
| amidopropyl | | | | | | | | | | | |
| betaine ¹ | | | | | | | | | | | |
| Sodium cocoyl | 6 | | | | | | | | | | |
| isethionate | | | | | | | | | | | |
| Sodium lauroyl | 2.5 | | | | | | | | | | |
| sarcosinate | | | | | | | | | | | |
| Rhamno di-lipid | | | 8.5 | | | | | | | | |
| mixture 1 ² | | | | | | | | | | | |
| Rhamno di-lipid | | | | | | 8.5 | | | | | |
| mixture 2 ³ | | | | | | | | | | | |
| Rhamno di-lipid | | | | | | | | | 8.5 | | |
| mixture 3 ⁴ | | | | | | | | | | | |
| Rhamno-mono- | | | | 8.5 | | | | | | | |
| lipid mixture 1 ⁵ | | | | | | | | | | | |
| Rhamno-mono- | | | | | 8.5 | | | | | | |
| lipid mixture 2 ⁶ | | | | | | | | | | | |
| Rhamno-mono- | | | | | | | | 8.5 | | | 8.5 |
| lipid mixture 3 ⁷ | | | | | | | | | | | |
| Rhamno-mono- | | | | | | | | | | | |
| lipid mixture 4 ⁸ | | | | | | | | | | | |
| Rhamno-mono- | | | | | | | 8.5 | | | | |
| lipid mixture 5 ⁹ | | | | | | | | | | | |
| Rhamno-mono- | | | | | | | | | | 8.5 | |
| lipid mixture 6 ¹⁰ | | | | | | | | | | | |
| Polyquaternium- | 0.3 | 0.3 | 0.3 | 0.3 | 0.3 | 0.3 | 0.3 | 0.3 | 0.3 | 0.3 | 0.3 |
| 10 | | | | | | | | | | | |
| Perfume | 1.1 | 1.1 | 1.1 | 1.1 | 1.1 | 1.1 | 1.1 | 1.1 | 1.1 | 1.1 | 1.1 |

| | | |
|---|---|---|
| Buffer | To pH 5.5-6.0 | To pH 6.8-7.2 |

| | |
|---|---|
| Water | QS |

| Component | C 4 | Inv 4 | C 5 | Ex 5 | C 6 | Ex 6 |
|---|---|---|---|---|---|---|
| Alkyl | 9.8 | 9.8 | 9.8 | 9.8 | 9.8 | 9.8 |
| amidopropyl | | | | | | |
| betaine ¹ | | | | | | |
| Sodium cocoyl | 6.0 | 6.0 | 6.0 | 6.0 | 6.0 | 6.0 |
| isethionate | | | | | | |
| Sodium lauroyl | 2.5 | 2.5 | 2.5 | 2.5 | 2.5 | 2.5 |
| sarcosinate | | | | | | |
| Rhamno di-lipid | | | | | | |
| mixture 1 ² | | | | | | |
| Rhamno di-lipid | 4.0 | | 2.0 | | 0.5 | |
| mixture 2 ³ | | | | | | |
| Rhamno di-lipid | | | | | | |
| mixture 3 ⁴ | | | | | | |
| Rhamno-mono- | | | | | | |
| lipid mixture 1 ⁵ | | | | | | |
| Rhamno-mono- | | 4.0 | | 2.0 | | 0.5 |
| lipid mixture 2 ⁶ | | | | | | |
| Rhamno-mono- | | | | | | |
| lipid mixture 3 ⁷ | | | | | | |
| Rhamno-mono- | | | | | | |
| lipid mixture 4 ⁸ | | | | | | |
| Rhamno-mono- | | | | | | |
| lipid mixture 5 ⁹ | | | | | | |
| Rhamno-mono- | | | | | | |
| lipid mixture 6 ¹⁰ | | | | | | |
| Polyquaternium- | 0.3 | 0.3 | 0.3 | 0.3 | 0.3 | 0.3 |
| 10 | | | | | | |
| Perfume | 1.1 | 1.1 | 1.1 | 1.1 | 1.1 | 1.1 |

| | |
|---|---|
| Buffer | To pH 6.8-7.2 |
| Water | QS |

¹ Mixture of chain lengths (Amphosol ® CG from Stepan)
² Rheance ® One brand rhamnolipids from Evonik (~50% active), Evonik Industries AG, Essen, Germany
³ Carfil ® Bio-RL1 rhamnolipid from Wanhua (~38% active), Wanhua Chemical Group Co., Ltd., Yantai, China
⁴ ReNuva ® RL-50 rhamnolipid from BioReNuva (~50% active), BioReNuva, Austin, TX, USA
⁵ Hydrolysate of Rheance ® One with HCl neutralization and EtOAc extraction (100% active)
⁶ Hydrolysate of Rheance ® One without post-hydrolysis workup (~100% active)
⁷ Hydrolysate of Carfil ® Bio-RL1 without post-hydrolysis workup (~100% active)
⁸ Hydrolysate of ReNuva ® RL-50 rhamnolipid without post-hydrolysis workup (~100% active)
⁹ Hydrolysate of Carfil ® Bio-RL1 with HCl neutralization and EtOAc extraction (~100% active)
¹⁰ Hydrolysate of ReNuva ® RL-50 ®with HCl neutralization and EtOAc extraction (~100% active)

The sulfate-free shampoo compositions listed in Table 2A were tested for lathering, conditioning and antimicrobial properties. The testing was conducted according to the methods described herein. The results of the testing are summarized in Table 2B.

TABLE 2B
Sulfate-free Shampoo Performance

| | Control | Control | | Inv1 | | | Inv2 | | | Inv3 | |
|---|---|---|---|---|---|---|---|---|---|---|---|
| Data | 1 | 2 | C1 | A | B | C2 | A | B | C3 | A | B |
| Lather Height (oil) | 8.3 | 3.0 | 2.3 | 9.5 | 8.0 | 2.0 | 9.0 | 9.5 | 2.5 | 8.0 | 9.0 |
| (cm) | | | | | | | | | | | |
| Lather Height (dirt) | 15.2 | 13.0 | 13.0 | 16.5 | 17.0 | 11.0 | — | 17.0 | 13.0 | — | 17.0 |
| (cm) | | | | | | | | | | | |
| Lather Creaminess | 4 | 0 | 0 | 5 | 4 | 0 | 5 | 8 | 0 | 5 | 5 |
| Speed to Lather | 7 | 5 | 7 | 7 | 7 | 6 | 7 | 7 | 5 | 7 | 8 |
| Slippery Feel - | 5 | 1 | 3 | 4 | 3 | 4 | 3 | 2 | 3 | 5 | 3 |
| Post Rinse | | | | | | | | | | | |
| Ease of Combing | 7 | 1 | 2 | 6 | 2 | 2 | 2 | 3 | 6 | 10 | 1 |
| MST log reduction - | <0.2 | <0.2 | <0.2 | ≥4.7 | 2.7 | <0.2 | >4.4 | >4.8 | <0.2 | >4.4 | 4.4 |
| Bacteria Day 2 | | | | | | | | | | | |
| MST log reduction | <0.2 | 0.2 | <0.2 | ≥4.7 | 3.9 | <0.2 | >4.4 | >4.8 | <0.2 | >4.4 | >4.8 |
| Bacteria Day 7 | | | | | | | | | | | |
| MST log reduction | 0.2 | <0.2 | 0.4 | 3.1 | 2.6 | <0.2 | 2.3 | >3.8 | <0.2 | 2.1 | >3.8 |
| Yeast/Mold Day 7 | | | | | | | | | | | |
| MST log reduction | 0.2 | 0.5 | 0.8 | ≥3.7 | 2.9 | 0.9 | 2.7 | >3.8 | 0.6 | 2.5 | ≥3.8 |
| Yeast/Mold Day 14 | | | | | | | | | | | |

| Data | C4 | Inv4 | C5 | Ex5 | C6 | Ex6 |
|---|---|---|---|---|---|---|
| Lather Height (oil) | 8.0 | 8.0 | 8.0 | 8.0 | 8.0 | 8.0 |
| (cm) | | | | | | |
| Lather Height (dirt) | — | — | — | — | — | — |
| (cm) | | | | | | |
| Lather Creaminess | 5 | 5 | 5 | 5 | 5 | 5 |
| Speed to Lather | 9 | 7 | 9 | 7 | 8 | 7 |
| Slippery Feel - | 3 | 4 | 4 | 5 | 5 | 5 |
| Post Rinse | | | | | | |
| Ease of Combing | 3 | 4 | 4 | 7 | 7 | 7 |
| MST log reduction - | <0.2 | >4.1 | <0.2 | 1.1 | <0.2 | <0.2 |
| Bacteria Day 2 | | | | | | |
| MST log reduction | <0.2 | >4.1 | <0.2 | 0.8 | <0.2 | <0.2 |
| Bacteria Day 7 | | | | | | |
| MST log reduction | 0.5 | 1.8 | 0.6 | 1.6 | 0.4 | 1 |
| Yeast/Mold Day 7 | | | | | | |
| MST log reduction | 1.7 | 2.1 | 1.1 | 1.5 | 1.2 | 1.5 |
| Yeast/Mold Day 14 | | | | | | |

As can be seen in Table 2B, inventive examples 1-4 yielded unexpectedly better multifunctional performance (i.e., better lather, conditioning, and antimicrobial properties) versus the comparative examples. In particular, it was not expected that using a mixture of rhamno-mono-lipids would produce this kind of improved performance.

Table 3A shows examples of shampoo compositions containing a sulfated surfactant and a rhamnolipid. The examples in Table 3A also include an amphoteric co-surfactant, cocamidopropyl betaine. The rhamno-mono-lipids used in the inventive examples were obtained by hydrolyzing the listed rhamno-di-lipid material and incorporating the unpurified reaction product into the composition. The sulfated surfactant is sodium lauryl sulfate (SLS).

The inventive compositions are designated Inv1, Inv2, Inv3 and Inv4. The comparative compositions are designated C1, C2, C3 and C4. The control composition in this example is an in-market, sulfated shampoo composition. The control provides a performance benchmark. Control Personal Cleansing Composition 1 can be formed by the following process. DI water is added to a mixing vessel and guar hydroxypropyl trimonium chloride is added to the mixing vessel, and the mixing continues until material has fully dissolved (with no visible particles remaining and batch is clear). After the Guar hydroxypropyl trimonium chloride has fully dissolved, the following materials are added to the mixing vessel: Sodium lauryl sulfate, Cocamidopropyl betaine, Citric acid, Panthenol, and Panthenyl ethyl ether. The vessel contents are mixed for at least 10 minutes. Perfume is then added and mixed in the mixture for at least 2 minutes. Citric Acid is used to titrate the mixture until a pH of 4.5 to 6.0 is reached. DI water is added to bring the final volume to 100%. The mixture is mixed for at least 10 minutes until homogeneity is achieved.

Comparative Personal Cleansing Compositions 1-3, 5, 6 and Inventive Personal Cleansing Compositions 1-3, 5, 6 can be formed by the following process. DI water is added to a mixing vessel and guar hydroxypropyl trimonium chloride is added to the mixing vessel, and the mixing continues until material has fully dissolved (with no visible particles remaining and batch is clear). After the Guar hydroxypropyl trimonium chloride has fully dissolved, the following materials are added to the mixing vessel: Sodium lauryl sulfate, Rhamnolipids, Cocamidopropyl betaine, Citric acid, Panthenol, and Panthenyl ethyl ether. The vessel contents are mixed for at least 10 minutes. Perfume is then added and mixed in the mixture for at least 2 minutes. Citric Acid is used to titrate the mixture until a pH of 4.5 to 6.0 is reached. DI water is added to bring the final volume to 100%.

The mixture is mixed for at least 10 minutes until homogeneity is achieved.

Comparative Personal Cleansing Composition 4 and Inventive Personal Cleansing Composition 4 can be formed by the following process. DI water is added to a mixing vessel and guar hydroxypropyl trimonium chloride is added to the mixing vessel, and the mixing continues until material has fully dissolved (with no visible particles remaining and batch is clear). After the Guar hydroxypropyl trimonium chloride has fully dissolved, the following materials are added to the mixing vessel: Rhamnolipids, Cocamidopropyl betaine, Citric acid, Panthenol, and Panthenyl ethyl ether. The vessel contents are mixed for at least 10 minutes. Perfume is then added and mixed in the mixture for at least 2 minutes. Citric Acid is used to titrate the mixture until a pH of 4.5 to 6.0 is reached. DI water is added to bring the final volume to 100%. The mixture is mixed for at least 10 minutes until homogeneity is achieved.

TABLE 3A
Sulfated Shampoo

| Component | Control | C1 | Inv1 | C2 | Inv2 | C3 | Inv3 | C4 | Inv4 |
|---|---|---|---|---|---|---|---|---|---|
| Sodium lauryl sulfate | 9.0 | 8.5 | 8.5 | 7.0 | 7.0 | 3.0 | 3.0 | | |
| Cocamidopropyl | 3.0 | 3.0 | 3.0 | 3.0 | 3.0 | 3.0 | 3.0 | 3.0 | 3.0 |
| betaine | | | | | | | | | |
| Rhamno di-lipid | | 0.5 | | 2.0 | | 6.0 | | 9.0 | |
| mixture 1 ¹ | | | | | | | | | |
| Rhamno-mono-lipid | | | 0.5 | | 2.0 | | 6.0 | | 9.0 |
| mixture 1 ² | | | | | | | | | |
| Guar hydroxypropyl | 0.1 | 0.1 | 0.1 | 0.1 | 0.1 | 0.1 | 0.1 | 0.1 | 0.1 |
| trimonium chloride ³ | | | | | | | | | |
| Panthenol | 0.1 | 0.1 | 0.1 | 0.1 | 0.1 | 0.1 | 0.1 | 0.1 | 0.1 |
| Panthenyl ethyl ether | 0.1 | 0.1 | 0.1 | 0.1 | 0.1 | 0.1 | 0.1 | 0.1 | 0.1 |
| Perfume | 1.0 | 1.0 | 1.0 | 1.0 | 1.0 | 1.0 | 1.0 | 1.0 | 1.0 |
| Citric acid | 0.3 | 0.3 | 0.3 | 0.3 | 0.3 | 0.3 | 0.3 | 0.3 | 0.3 |

| | |
|---|---|
| Buffer | To pH 4.5-6.0 |
| Water | QS |

| Component | C5 | Inv5 | C6 | Inv6 |
|---|---|---|---|---|
| Sodium lauryl sulfate | 9.0 | 9.0 | 9.0 | 9.0 |
| Cocamidopropyl | 3.0 | 3.0 | 3.0 | 3.0 |
| betaine | | | | |
| Rhamno di-lipid | 0.5 | | 2.0 | |
| mixture 1 ¹ | | | | |
| Rhamno-mono-lipid | | 0.5 | | 2.0 |
| mixture 1 ² | | | | |
| Guar hydroxypropyl | 0.1 | 0.1 | 0.1 | 0.1 |
| trimonium chloride ³ | | | | |
| Panthenol | 0.1 | 0.1 | 0.1 | 0.1 |
| Panthenyl ethyl ether | 0.1 | 0.1 | 0.1 | 0.1 |
| Perfume | 1.0 | 1.0 | 1.0 | 1.0 |
| Citric acid | 0.3 | 0.3 | 0.3 | 0.3 |

| | |
|---|---|
| Buffer | To pH 4.5-6.0 |
| Water | QS |

¹ Rheance ® One from Evonik (~50% active)
² Hydrolysate of Rheance ® One without post-hydrolysis workup (~100% active)
³ Jaguar Excel ™ from Solvay Novecare, Cranbury, NJ, USA

The sulfated shampoo compositions listed in Table 3A were tested for lathering, conditioning and antimicrobial properties. The testing was conducted according to the methods described herein. The results of the testing are summarized in Table 3B.

TABLE 3B
Sulfated Shampoo Performance

| Component | Control | C 1 | Inv 1 | C 2 | Inv 2 | C 3 | Inv 3 | C 4 | Inv 4 |
|---|---|---|---|---|---|---|---|---|---|
| Lather Blender | 9.0 | 8.5 | 9.0 | 8.5 | 9.0 | 5.5 | 8.0 | 2.0 | 5.5 |
| Height (oil) (cm) | | | | | | | | | |
| Lather Blender | 5 | 5 | 5 | 5 | 5 | 3 | 5 | 0 | 2 |
| Creaminess | | | | | | | | | |
| Speed to Lather | 7 | 8 | 8 | 7 | 9 | 7 | 4 | 6 | 4 |
| Slippery Feel - | 2 | 3 | 2 | 2 | 4 | 3 | 2 | 2 | 2 |
| Post Rinse | | | | | | | | | |
| Ease of Combing | 0 | 4 | 1 | 2 | 1 | 2 | 2 | 1 | 1 |
| MST log reduction - | <0.2 | <0.2 | 0.6 | 1 | >4.1 | <0.2 | >4.1 | 0.8 | >4.1 |
| Bacteria Day 2 | | | | | | | | | |
| MST log reduction | <0.2 | <0.2 | >4.1 | <0.3 | >4.1 | <0.2 | >4.1 | 2.5 | >4.1 |
| Bacteria Day 7 | | | | | | | | | |
| MST log reduction | 0.4 | 1.9 | 1 | >3.6 | >3.6 | <0.2 | >3.6 | 1.2 | >3.6 |
| Yeast/Mold Day 7 | | | | | | | | | |
| MST log reduction | 0.6 | 0.6 | 1.3 | >3.6 | >3.6 | 0.7 | >3.6 | 2.7 | >3.6 |
| Yeast/Mold Day 14 | | | | | | | | | |

| Component | C 5 | Inv5 | C6 | Inv6 |
|---|---|---|---|---|
| Lather Blender | 9.0 | 9.5 | 9.0 | 9.5 |
| Height (oil) (cm) | | | | |
| Lather Blender | 5 | 5 | 5 | 5 |
| Creaminess | | | | |
| Speed to Lather | 9 | 8 | 9 | 9 |
| Slippery Feel - | 2 | 2 | 2 | 2 |
| Post Rinse | | | | |
| Ease of Combing | 1 | 1 | 1 | 1 |
| MST log reduction - | <0.2 | 0.6 | <0.2 | >4.1 |
| Bacteria Day 2 | | | | |
| MST log reduction | <0.2 | >4.1 | <0.2 | >4.1 |
| Bacteria Day 7 | | | | |
| MST log reduction | <0.2 | 1.0 | <0.2 | >3.6 |
| Yeast/Mold Day 7 | | | | |
| MST log reduction | 0.0 | 1.3 | 0.2 | >3.6 |
| Yeast/Mold Day 14 | | | | |

As can be seen in Table 3B, the inventive examples yielded unexpectedly better multifunctional performance (i.e., better lather, conditioning, and antimicrobial properties) versus the comparative examples. In particular, it was not expected that a mixture of rhamno-mono-lipids would exhibit improved antimicrobial and lathering performance.

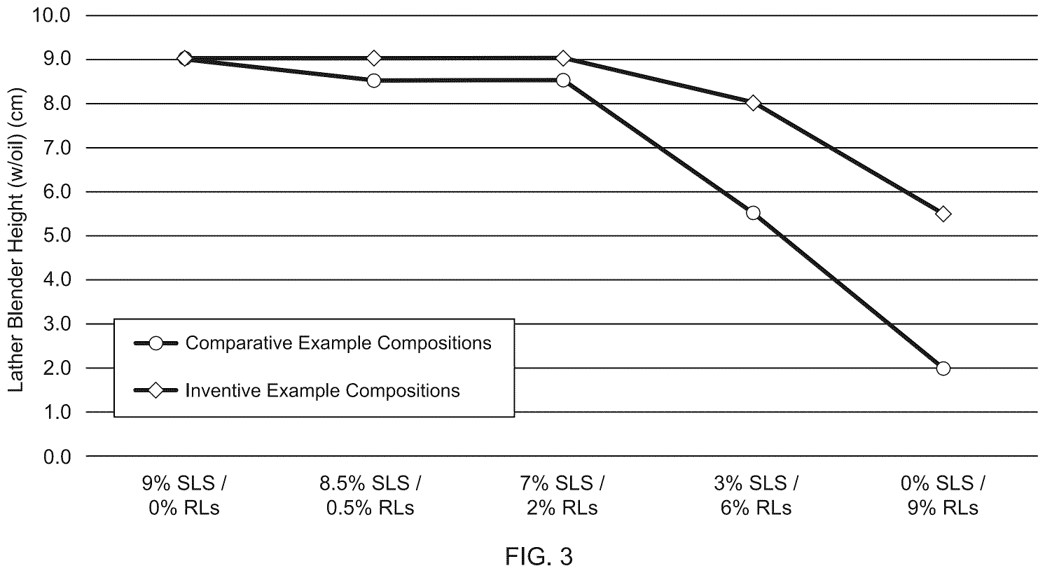

FIG. 3 shows a comparison of the lather performance of the inventive compositions versus the comparative compositions in Table 3A. Surprisingly, as can be seen in FIG. 3, combining a relatively low amount of SLS with the rhamno-mono-lipid delivered much better lather performance than when the commercially available rhamno-di-lipid material was used. This is desirable because it enables the formulation of a milder shampoo that delivers good lather.

### Example 3—Rhamnolipid Mixture

This example demonstrates the surprising lather benefit obtained by mixing the new multifunctional rhamnolipids with a conventional rhamnolipid surfactant. In this example, a mixture of various rhamnolipid surfactant solutions (pH 7) were tested to measure lather volume.

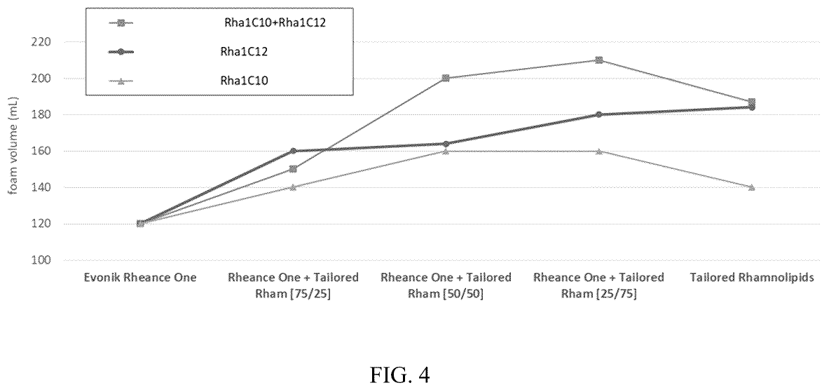

Lather volume is an important characteristic of a surfactant or shampoo because a consumer typically associates the volume of lather produced by a shampoo with its cleaning performance. In addition, some consumers enjoy the aesthetics of a high lather volume. The results of the testing are illustrated in FIG. 4.

The surfactant solutions in this example were made by mixing the rhamnolipids at the ratios shown in FIG. 4 and then adding water to form a solution of 1.5% rhamnolipid. The amount of commercially available rhamnolipid surfactant and tailored rhamno-mono-lipid were varied from 0% to 100%, as shown in FIG. 4. Three tailored rhamnolipid solutions were used to demonstrate the difference that the carbon chain length can make in the lipid tail. As can be seen in FIG. 4, when the ratio of conventional rhamnolipid to tailored rhamnolipid is high (approximately 3:1 or more), the foam volume behaves as one would expect, which is roughly an average of the individual foam volumes of the C10 and C12 rhamnolipids. However, at lower ratios of conventional rhamnolipid to tailored rhamnolipid (e.g., less than about 3:1 to 1:10 or more), the foam volume for the combination of chain lengths is greater than either of the individual chain lengths, which suggests a synergistic effect on foam volume.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care composition, comprising:
a) a mono-rhamno-mono-lipid of structure I: 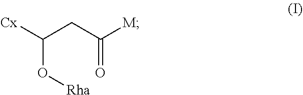
b) a di-rhamno-mono-lipid of structure II: 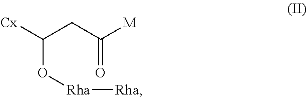
wherein:
Rha is rhamnose,
is a C4-C22 alkyl, aryl, heteroalkyl, heteroaryl, unsaturated alkenyl, or unsaturated heteroalkenyl, and
M is a OH, alkyl, heteroalkyl, aryl, heteroaryl, hetero arylalkyl, arylalkyl, tauryl, O-X+, wherein X+is a cation, or O-R1, wherein R1 is selected from an alkyl, branched alkyl, and cyclic alkyl, and stereoisomers thereof; and
c) a carrier.

2. The personal care composition of claim 1, wherein a ratio of the total amount of mono-rhamno-mono-lipid to the total amount of di-rhamno-mono-lipid is 10:1 to 1:10.

3. The personal care composition of claim 1, wherein the composition provides a 2 log or greater reduction in bacteria according to the Microbial Susceptibility Test.

4. The personal care composition of claim 1, wherein composition provides a 2 log or greater reduction in yeast and/or mold level according to the Microbial Susceptibility Test.

5. The personal care composition of claim 1, wherein the composition has a Lather Height of greater than 2.5 cm according to the Blender Lather Height (oil) method.

6. The personal care composition of claim 1, wherein the composition has a Lather Height of greater than 13 cm according to the Blender Lather Height (dirt) method.

7. The personal care composition of claim 1, wherein the composition further comprises a beta hydroxy fatty acid having a formula III:

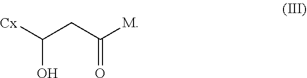

8. The personal care composition of claim 7, wherein the beta hydroxy fatty acid is selected from 3-hydroxytetradecanoic acid, 3-hydroxyhexadecanoic acid, 3-hydroxydeceneoic acid, 3-hydroxytetradecenoic acid, 3-hydroxydodecanoic acid, 3-hydroxydodecenoic acid, 3-hydroxyoctanoic acid, 3-hydroxydodec-dienoic acid, and 3-hydroxydecaneoic acid.

9. The personal care composition of claim 1, wherein at least one of the mono-rhamno-mono-lipid of structure I and di-rhamno-mono-lipid of structure II is present at 25 wt % or more, based on the total weight of rhamnolipids.

10. The personal care composition of claim 1, wherein the total amount of the mono-rhamno, mono-lipid of structure I and the di-rhamno, mono-lipid of structure II is 80 wt % or more, based on the total weight of rhamnolipids.

11. The personal care composition of claim 1, wherein the cation is selected from Na+, K+, Li+, Cs+, +NH3R2; +NH2R2R3; +NHR2R3R4, and +NR2R3R4R5, wherein R2, R3,R4, and R5 are each independently selected from an alkyl, branched alkyl, and cyclic alkyl.

12. The personal care composition of claim 1, wherein the composition comprises less than 25 wt % of a rhamno di-lipid, based on the total weight of rhamnolipids in the composition.

13. The personal care composition of claim 1, further comprising an additional antimicrobial agent selected from benzoates, salicylates, sorbates, phenoxyethanol, parabens, caprylyl glycol, substituted ureas, hydantoin derivatives, and combinations thereof.

14. The personal care composition of claim 1, wherein the mono-rhamno mono-lipid is selected from 3-(((2R,3R,4R,5R,6S)-3,4,5-trihydroxy-6-methyltetrahydro-2H-pyran-2-yl)oxy)hexadecanoic acid, 3-(((2R,3R,4R,5R,6S)-3,4,5-trihydroxy-6-methyltetrahydro-2H-pyran-2-yl)oxy)octenoic acid, 3-(((2R,3R,4R,5R,6S)-3,4,5-trihydroxy-6-methyltetrahydro-2H-pyran-2-yl)oxy)decenoic acid, 3-(((2R,3R,4R,5R,6S)-3,4,5-trihydroxy-6-methyltetrahydro-2H-pyran-2-yl)oxy)octanoic acid, 3-(((2R,3R,4R,5R,6S)-3,4,5-trihydroxy-6-methyltetrahydro-2H-pyran-2-yl)oxy) tetradecanoic acid, 3-(((2R,3R,4R,5R,6S)-3,4,5-trihydroxy-6-methyltetrahydro-2H-pyran-2-yl)oxy)decanoic acid, 3-(((2R,3R,4R,5R,6S)-3,4,5-trihydroxy-6-methyltetrahydro-2H-pyran-2-yl)oxy) dodecanoic acid, 3-(((2R,3R,4R,5R,6S)-3,4,5-trihydroxy-6-methyltetrahydro-2H-pyran-2-yl)oxy)dodecen-dienoic acid, and stereoisomers thereof.

15. The personal care composition of claim 1, wherein the di-rhamno mono-lipid is selected from 3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)hexadecanoic acid, 3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)tetradecanoic acid, 3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)decenoic acid, 3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)tetradecenoic acid, 3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)decanoic acid, 3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)octanoic acid, and 3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl) oxy) dodecanoic acid, and stereoisomers thereof.

16. The personal care composition of claim 1, wherein the composition is substantially free of sulfated surfactants.

17. The personal care composition of claim 1, further comprising a surfactant selected from an amphoteric surfactant, a zwitterionic surfactant, a non-ionic surfactant, a cationic surfactant, an anionic surfactant or a combination of these.

18. A personal care composition, comprising:
a) a mono-rhamno-mono-lipid of structure I: 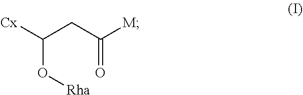
b) a di-rhamno-mono-lipid of structure II: 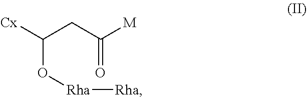
wherein:
Rha is rhamnose,
Cx is a C4-C22 alkyl, aryl, heteroalkyl, heteroaryl, unsaturated alkenyl, or unsaturated heteroalkenyl, and
M is a OH, alkyl, heteroalkyl, aryl, heteroaryl, hetero arylalkyl, arylalkyl, tauryl, O-X+, wherein X+is a cation, or O-R1, wherein R1 is selected from an alkyl, branched alkyl, and cyclic alkyl, and stereoisomers thereof;
c) a rhamno-di-lipid; and
d) a carrier.

19. The personal care composition of claim 18, wherein a ratio of rhamno-di-lipid to a total amount of rhamn-mono-lipid is less than 3:1.

20. The personal care composition of claim 19, wherein the composition exhibits synergistic lather volume compared to a control composition comprising a ratio of rhamno-di-lipid to a total amount of rhamn-mono-lipid of 3:1.
